(19) United States
(12) Patent Application Publication
(10) Pub. No.: US 2023/0400455 A1
(43) Pub. Date: Dec. 14, 2023

(54) ELECTROCHEMICAL SENSOR

(71) Applicant: University of Strathclyde, Glasgow (GB)

(72) Inventors: Damion K. Corrigan, Glasgow (GB); Vincent James Vezza, Glasgow (GB)

(21) Appl. No.: 18/033,838

(22) PCT Filed: Oct. 26, 2021
(86) PCT No.: PCT/GB2021/052781

§ 371 Date: Apr. 26, 2023

(30) Foreign Application Priority Data
Oct. 27, 2020 (GB) 2017047.8

(51) Int. Cl. G01N 33/543; G01N 33/569

(52) U.S. Cl. CPC G01N 33/5438; G01N 33/56983; G01N 2610/00; G01N 2333/165

(57) ABSTRACT

The present disclosure relates to an electrochemical sensor, which employ fluoro organothiol or fluoro organosilane molecules in the formation of self-assembled monolayer (SAM) for use in diagnosis tests. There is also provided methods of testing a patient sample using the electrochemical sensor as disclosed.

### FIELD OF THE DISCLOSURE

The present disclosure relates to an electrochemical sensor for use in diagnosis tests. There is also provided methods of testing a patient sample using the electrochemical sensor as disclosed.

### BACKGROUND OF THE DISCLOSURE

Electrochemical biosensors are a promising route to realising rapid and sensitive detection of a large range of pathogens and clinically important biomarkers¹. The most well-known example is the glucose biosensor (most commonly an amperometric sensor), which is in widespread use for the home testing of blood glucose levels and which serves diabetic patients so well in the routine monitoring of blood sugar levels². Numerous other biosensors have been developed which operate by a range of principles³, including, cyclic voltammetry (CV)⁴, linear sweep voltammetry (LSV)⁵, electrochemical impedance spectroscopy (EIS)⁶ and differential pulse voltammetry (DPV)⁷. EIS involves a measurement setup where a small AC excitation potential is imposed at the working electrode (often under open circuit potential) and the resulting current response of the electrochemical cell is measured. Various parameters associated with the cell and its response can be extracted from the EIS response, and these include the solution resistance (R_{S}), the double layer capacitance (C_{DL}), the charge transfer resistance (R_{CT}) and the Warburg impedance (W)⁸. The double layer capacitance and the charge transfer resistance have been shown to be particularly effective for the label-free monitoring of binding at biologically functionalised electrode surfaces. These techniques enable the sensitive and specific measurement of DNA and protein biomarkers which has been shown repeatedly in the literature⁹.

For many electrochemical biosensors, like those described above, surface functionalisation and attachment chemistry play a major role in sensor design and performance^{10,11}. For gold sensors, the attachment of biological molecules often takes place through use of gold-thiol attachment and more specifically through the formation of self-assembled monolayers (SAMs)¹². SAMs serve the dual purpose of blocking the electrode surface from non-specific binding of proteins, cells and other components in a sample medium and can ensure correct orientation of the bio-recognition element (e.g. DNA sequence, antibody or enzyme)¹³. Self-assembled layers are often formed by incubation of gold surfaces with solutions of thiolated biomolecules and can contain single molecule (monolayer) or multi component forms where additional complexity is introduced in order to ensure adequate orientation of the receptor and good resistance to surface fouling.

### SUMMARY OF THE DISCLOSURE

The present disclosure is based on work conducted by the investigators into the development of sensors for diagnostic use, which employ fluoro organothiol or fluoro organosilane molecules in the formation of SAMs. In one embodiment SAMs are formed using 1H,1H,2H,2H-Perfluorodecanethiol (PFDT) on sensor surfaces which is then bio-functionalised with a biological agent. PFDT spontaneously forms a dense hydrophobic SAM on gold surfaces, and reduces surface biofouling¹⁴. PFDT has been previously used to enhance performance of organic transistors¹⁵ and to reversibly organise DNA onto a micro-patterned substrate¹⁶.

In a first teaching, the present disclosure provides an electrochemical biosensor for use in detecting a target analyte, the sensor comprising:
at least one detection electrode comprising a surface coated with a self-assembled monolayer (SAM), wherein the SAM comprises, consists essentially of, or consists of a hydrofluorocarbon or fluorocarbon molecule bound to the surface of the electrode through a reactive sulphur or silicon group present on the hydrofluorocarbon or fluorocarbon.

A hydrofluorocarbon is an organic compound, which contains fluorine and hydrogen atoms. A fluorocarbon is a compound in which all the C—H bonds have been replaced by C—F bonds. The hydrofluorocarbon or fluorocarbon molecule may take the form of a linear, branched or cyclic alkane, alkene or alkyne molecule having a single or multiple reactive sulphur or silicon groups.

In one embodiment, the reactive sulphur group(s) may be a thiol, as such the molecule is a fluoro organothiol. In one embodiment, the reactive silicon group(s) may be a silane, as such the molecule is a fluoro organosilane.

In one embodiment, the fluorocarbon molecule is a linear fluoro alkanethiol, or fluoro alkanesilane.

Exemplary compounds suitable for use in the present disclosure include:
1H, 1H,2H,2H-Perfluorodecanethiol
3,3,4,4,5,5,6,6,7,7,8,8,8-Tridecafluoro-1-octanethiol
3,3,4,4,5,5,6,6,7,7,8,8,9,9,10,10,10-Heptadecafluoro-1-decanethiol
3,3,4,4,5,5,6,6,6-Nonafluoro-1-hexanethiol
2,2,2-Trifluoroethanethiol
1H,1H,2H,2H-Perfluorooctyltriethoxysilane
1H,1H,2H,2H-Perfluorodecyltriethoxysilane; and
1H,1H,2H,2H-Perfluorododecyltrichlorosilane.

In one embodiment the compound for use in forming a SAM is 1H,1H,2H,2H-Perfluorodecanethiol

The electrodes of the present disclosure may be made of any suitable electrically conductive material, which may be coated by the hydrofluorocarbon or fluorocarbon molecules as described herein. Suitable materials include glassy carbon; metal oxide; conducting polymer; and noble metals including gold, ruthenium, rhodium, palladium, platinum and silver. In one particular embodiment, the electrode is gold.

A self-assembled monolayer (SAM) is a self-organized layer of typically amphiphilic molecules in which one end of the molecule shows a specific affinity for a substrate material. SAM molecules can include a head group that anchors the molecule to the substrate, as well as a tail portion or functional group at the terminal end. SAM layers can be formed by the chemisorption of head groups, such as reactive sulphur (e.g. thiol) and silicon (e.g. silane) groups onto a substrate material from the vapor or liquid phase. The tail portion group of the SAMs according to the present disclosure make use of the known, fluorous effect¹⁷ and are hydrophobic in nature, allowing capture or physisorption of a variety of biological agents as will be described in more detail herein.

Advantageously, the hydrophobic nature of the SAM not only allows a relevant biological agent to be captured by the SAM, but also provides a barrier layer which serves to reduce fouling and/or interference from other materials, such as proteins, cells and other molecules, which may be present with a sample. A sample may be obtained from a subject and may blood, saliva or any other suitable biological fluid, such as urine, semen, or tissue sample. Alternatively, the sample may be an environmental sample, for example a water sample, soil sample, or even a plant sample.

In a further teaching, the present disclosure further provides an electrochemical biosensor as described in the first teaching and embodiments, further comprising a biological agent captured, such as by physisorption, by the SAM layer coated on the surface of the electrode.

In a further teaching, the present disclosure provides a method of making an electrochemical biosensor as described in the first or further teachings and embodiments described above, the method comprising: forming a SAM on a surface of at least one detection electrode, by contacting the surface of the at least one detection electrode with a solution comprising an organic solvent and a hydrofluorocarbon or fluorocarbon molecule as described hereinabove,
allowing the solvent to evaporate and the SAM to form on the surface of the at least one detection electrode; and
optionally subsequently contacting the SAM coated electrode with a solution comprising the biological agent and allowing the biological agent to be captured by the SAM layer coated on the electrode.

In some embodiments of the present disclosure, the electrochemical biosensors further comprise at least one reference and/or counter electrode, which may be electrically coupled to said at least one detection electrode, e.g. via a measurement system or connections for connecting to a measurement system. The electrodes are typically on a substrate and may, for example, be provided in the form of screen printed electrodes; microelectrodes; on a printed circuit board; FETS/OFETS and the like. The electrochemical biosensors may comprise, or be provided with connections for connecting to, a measurement system. The measurement system may be configured to apply an electrical signal between the at least one detection electrode and the at least one reference and/or counter electrode. The measurement system may be configured to measure an electrical response resulting from the detection of an analyte by the biological agent. The measurement system may be configured to perform impedance, voltammetric, or amperometric measurements. The measurement system may be configured to perform electrochemical impedance spectroscopy (EIS).

An electrochemical biosensor in accordance with the present disclosure may be used detect a target analyte which is capable of binding, typically specifically binding, to the biological agent captured by the sensor. The target analyte may be a chemical (such as a hormone, narcotic or pollutant) or biological molecule, such as a peptide, protein, glycoprotein, enzyme, glycolipid, cell surface receptor, cytokine, antibody, nucleic acid or the like. The target analyte may be free within the sample being analyzed, or may still be part of a cell, cell membrane, virus coat etc in which the target analyte, such as biological molecule, is normally found in situ. Binding between the biological agent and the target analyte involves the attractive binding (i.e. non-repelling) of two or more species held together by attractive forces. Such binding comprises an interaction between the biological agent and the target analyte that pulls (or draws) the biological agent and target analyte in a sample together. Binding includes, but is not limited to covalent interactions; electrostatic interactions; ion-ion interactions, for example between attractive or opposite charges; dipole interactions; ion-dipole interactions; hydrogen bonding interactions; van der Waals interactions; pi-stacking interactions; the sharing of electron density or combinations thereof.

The electrochemical sensors of the present disclosure provide a signal transduction method, whereby any binding of the target analyte to the biological agent may be converted into a signal for processing and/or display. The biological agents may be similar to the target analytes and may include proteins, enzymes, antibodies, nucleic acids, etc. The biosensors can be configured to use specific chemical interaction properties (such as an enzyme and its substrate) or molecular recognition mechanisms (such as a protein binding to a receptor or antibody binding to an antigen) to identify target analytes. Biosensors can use the electrode to transform an electrical signal resulting from the detection of an analyte by the biological agent into a different signal that can be addressed by optical, electronic or other means.

In one embodiment, impedance, voltammetric, or amperometric measurements may be performed in order to detect the signal, or a change in signal, by the electrochemical sensor. In one embodiment, the electrochemical detection of a target analyte is carried out by electrochemical impedance spectroscopy (EIS). In this manner, the electrochemical biosensor of the present disclosure transduces changes in the interfacial properties between the electrode and an electrolyte induced by the biological macromolecule binding to the target analyte to an electrical signal. Typically, a redox pair, such as K₃[Fe(CN)₆]/K₄[Fe(CN)₆] may be used as a redox indicator for the electrode kinetics at the interface. Sensors based on the use of EIS detection are label-free and, thus, possess advantages of low cost, simplicity and ease of miniaturization. EIS is particularly useful because it is sensitive to surface interactions and quantifies the interfacial charge transfer resistance (R_{CT}) that is associated with charged redox probes. R_{CT} is strongly affected by changes in charge distributions near the electrode-solution interface in a sample solution, which results in surface sensitivity.

The biosensors of the present disclosure may find application in many different diagnostic applications, including detection of infectious agents, such as bacteria, viruses (including as influenza, SARS-COV2) and the like; molecules such as IL-16 and procalcitonin associated with sepsis; cardiac biomarkers such as troponin; and liver biomarkers, for example. Advantageously, the principles of detection using the sensors of the present invention have broad applicability due to the relative ease of manufacture and speed of detection.

In one non-limiting embodiment, the present disclosure is directed to the detection of COVID-19. In this example, the biological macromolecule, which is captured by the electrochemical biosensor is ACE-2, which has been identified as an entry receptor for SARS-CoV-2, the virus responsible for COVID-19. Thus, in an embodiment, an electrochemical biosensor in accordance with the present disclosure, wherein the biological macromolecule is ACE-2, may used in the detection of SARS-CoV-2, such as COVID 19. Alternatively, a COVID 19 specific antibody¹⁸, such as an antibody, which is specifically able to bind the spike protein of COVID 19 may be used as the biological macromolecule. In this manner, the target analyte to be detected is the virus or a coat protein of SARS-CoV-2 or more specifically COVID 19.

The chemical terminologies as used herein have their standard meanings known in the art, in accordance with the IUPAC Goldbook, unless explicitly stated. Unless the context clearly requires otherwise, throughout the description and the claims, the words “comprise”, “comprising” and the like, are to be construed in an inclusive sense as opposed to an exclusive or exhaustive sense, that is to say, in the sense of “including, but not limited to”.

### DETAILED DESCRIPTION

The present disclosure will now be further described by way of example and with reference to the following figures, which show:

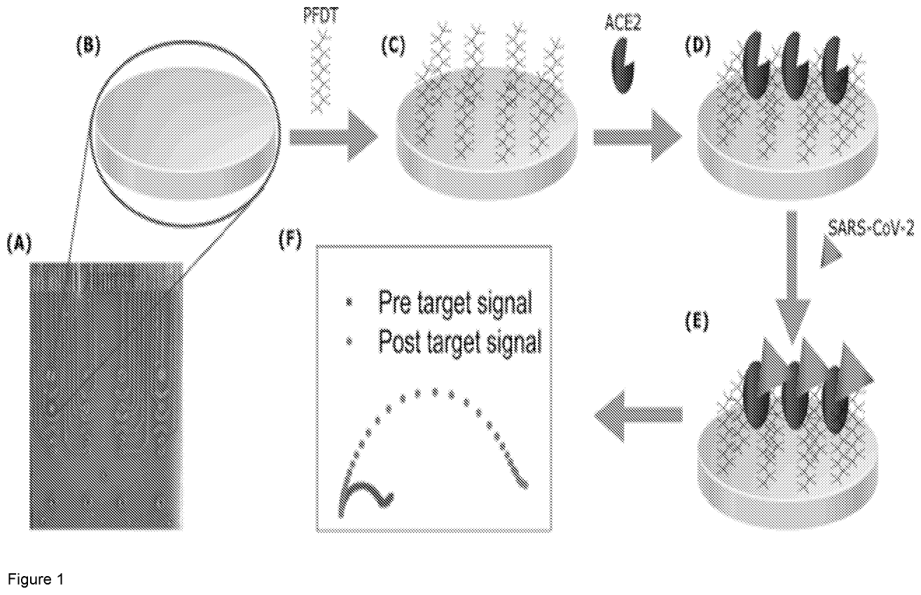

FIG. 1. (A) Image of the 8×Au working electrode PCB based sensor array with on chip Au counter and reference electrodes. (B, C, D, E) Representations of the Au sensor surface in the following states: clean (B), PFDT functionalised and (C) ACE2 functionalised (D) and with binding of SARS-CoV-2 spike protein or inactivated virus (E). (F) Example Nyquist plots showing the signal from an ACE2 functionalised sensor (black) and following exposure to recombinant SARS-CoV-2 spike protein (red).

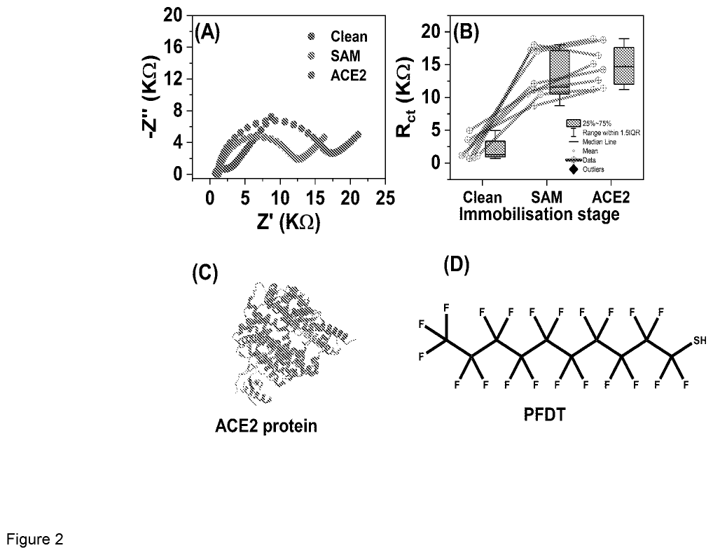

FIG. 2. (A) Example Nyquist plots from a representative electrode following cleaning (black), PFDT functionalisation (red) and ACE2 incubation (blue). (B) Box plot showing Rd values through the three stages of electrode functionalisation (cleaning, SAM formation and ACE2 immobilisation). (C) Protein structure of ACE2(1R42)¹⁹. (D) Structural formula of PFDT.

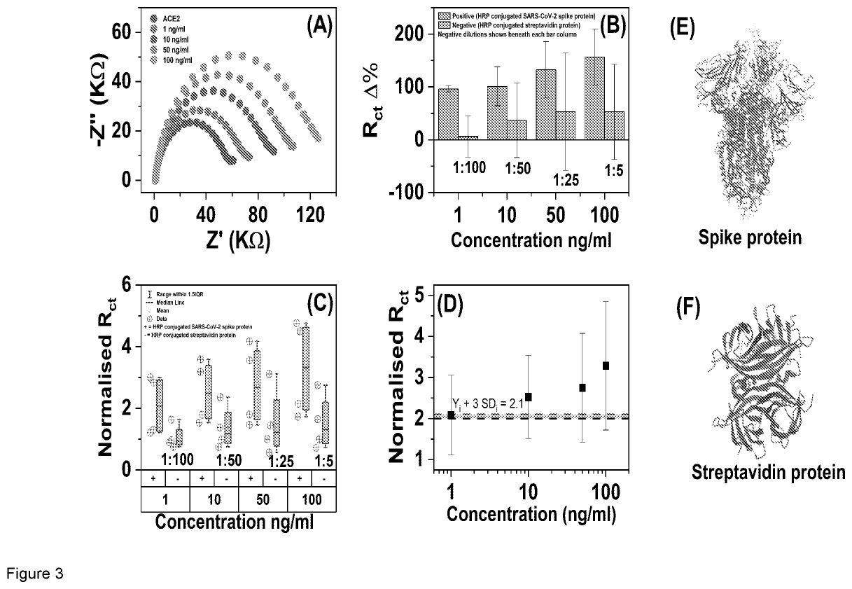

FIG. 3. (A) Box plot showing normalised Rd values for ACE2 functionalised electrodes versus HRP conjugated SARS-CoV-2 spike protein solutions and HRP conjugated streptavidin solutions. (B) Nyquist plot showing the impedimetric response to increasing HRP conjugated SARS-CoV-2 spike protein. (C) Bar chart showing HRCT % change in response to addition of HRP conjugated SARS-CoV-2 spike and HRP conjugated streptavidin proteins. (D) Dose response curve for HRP conjugated SARS-CoV-2 spike protein. (E) Protein structures of SARS-CoV-2 spike protein (6XM4)²⁰ and streptavidin(4BX5)²¹.

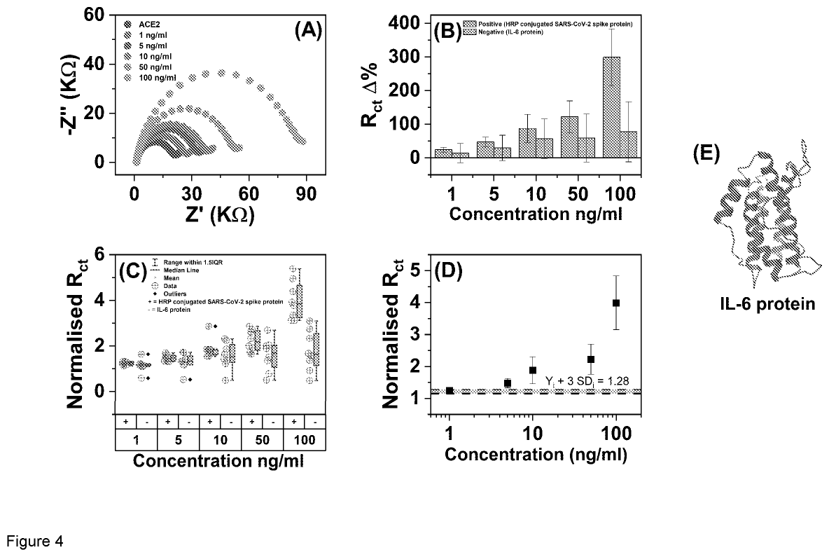

FIG. 4. (A) Nyquist plot showing the impedimetric response to increasing HRP conjugated SARS-CoV-2 spike protein. (B) Bar chart showing ΔR_{CT} % change in response to addition of HRP conjugated SARS-CoV-2 spike and IL-6. (C) Box plot showing normalised Rd values for ACE2 functionalised electrodes versus HRP conjugated SARS-CoV-2 spike protein solutions and IL-6 solutions. (D) Dose response curve for HRP conjugated SARS-CoV-2 spike protein. (E) Protein structure of IL-6 (2IL6)²⁴.

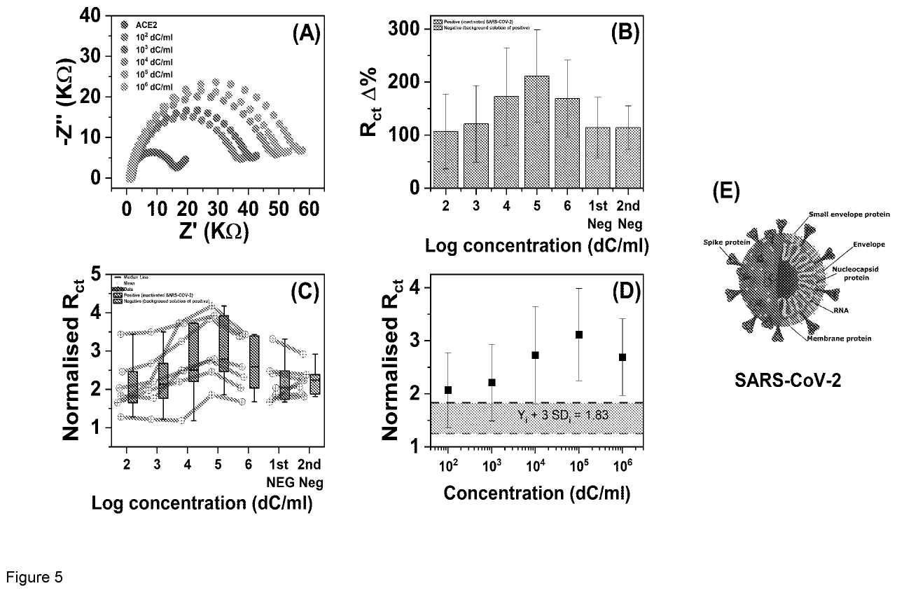

FIG. 5. (A) Nyquist plot showing the impedimetric response to increasing concentrations of inactivated SARS-CoV-2 virus. (B) Bar chart showing ΔR_{CT} % change in response to addition of negative and positive samples of inactivated SARS-CoV-2. (C) Box plot showing normalised Rd values for ACE2 functionalised electrodes versus positive and negative samples of inactivated SARS-CoV-2. (D) Dose response curve for inactivated SARS-CoV-2. (E) SARS-CoV-2 structure (Adapted from an image by: Maya Peters Kostman for the Innovative Genomics Institute. https://creativecommons.org/licenses/by-nc-sa/4.0/legalcode).

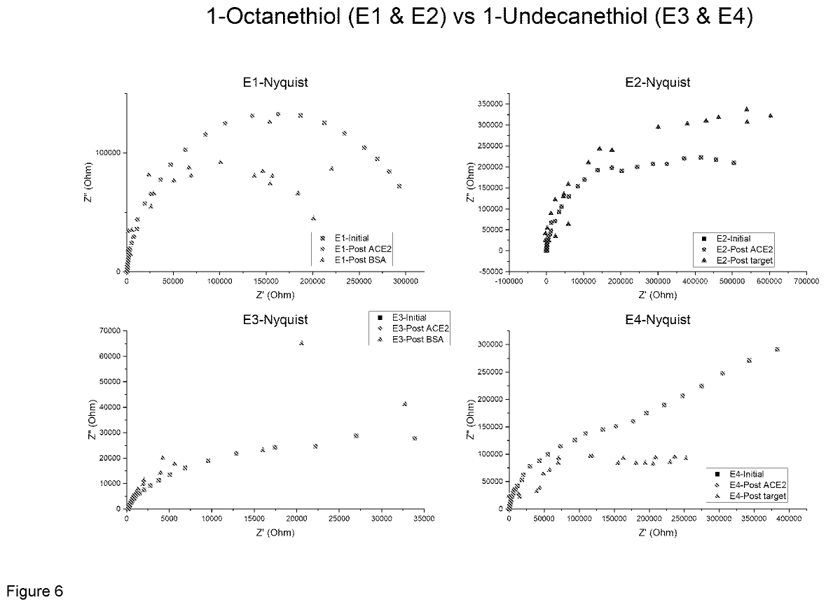

FIG. 6. Showing impedance changes like those observed for PFDT modified electrodes, characteristic of increasing charge transfer resistance upon spike protein binding were not observable when the underlying SAM layer was composed of 1-octanethiol or 1-undecanethiol.

### MATERIALS AND METHODS

Abbreviations PFDT, 1H, 1H, 2H, 2H-perfluorodecanethiol; ACE2, Angiotensin converting enzyme 2; IL-6, Interleukin-6; SARS-CoV-2, Severe acute respiratory syndrome coronavirus 2; HRP, Horseradish peroxidase; EIS, Electrochemical impedance spectroscopy; ARDS, Acute respiratory distress syndrome; PCB, Printed circuit board; PBS, Phosphate-buffered saline; SAM, Self-assembled monolayer; Rd, Charge transfer resistance; OCP, open circuit potential; IQR, inter quartile range.

Chemicals. K₃[Fe(CN)₆], K₄[Fe(CN)₆ ], 1H,1H,2H,2H-perfluorodecanethiol, KOH and H₂O₂ 30% (v/v) were obtained from Sigma-Aldrich. Toluene was obtained from Fisher Scientific UK Ltd (Loughborough, UK). Deionised water (5.00 μS/cm @ 25° C.) was purchased from Scientific Laboratory Supplies Limited (Nottingham, UK). Inactivated SARS-CoV-2 and negative control obtained from Randox laboratories Ltd (Crumlin, UK). ACE2 was purchased from Abcam (Cambridge, UK), HRP conjugated spike protein was purchased from The Native Antigen Company (Oxford, UK) and HRP conjugated streptavidin was purchased as part of an IL-6 diagnostics kit from Bio-techne (Abingdon, UK).

Preconditioning. SEPI BIOTIP multichannel electrode PCB platform (biotip ltd, Bath, UK) were cleaned according to the supplied protocol. This consisted of a 15-minute submersion in a solution of 50 mM KOH in H₂O₂ 30% (v/v) at room temperature. The PCB was then rinsed with DI water and dried using compressed air. The PCB was then electrochemically cleaned by submerging in 50 mM KOH (DI water as solvent) with an external platinum counter electrode (Metrohm, Runcorn, UK) and 3M NaCl Ag/AgCl reference electrode (IJ Cambria, Llanelli, UK). Cyclic voltammetry was performed on all working electrodes on the PCB using the following parameters: potential window was −1.2 to 0.6 V, scan rate of 0.1 V/s and 15 scans per electrode. The PCB was then rinsed with DI water and dried again using compressed air. All electrochemical measurements were performed using a PalmSens4 potentiostat and the accompanying PSTrace software, both supplied by Palmsens BV (Houten, Netherlands).

Fluorous SAM and ACE2 immobilisation. The SAM solution was prepared by magnetically stirring toluene and adding PFDT until a 1 mM solution was formed. Stirring aids in dispersing the PFDT throughout the solution. Fluorocarbons can have low miscibility in organic solvents and have a propensity for self-interaction forming separate phases via the fluorous effect¹⁷. The PCBs were orientated horizontally in a small glass petri dish and the PFDT solution added to cover the PCB with excess solution. Toluene evaporates quickly, therefore having excess solution and a film covering reduced evaporative losses. The PCBs were incubated overnight at room temperature, then rinsed with DI water (10 second water bottle flow per electrode) and dried with compressed air. All work with toluene was performed in a suitable fume hood with proper halogenated solvent waste disposal routes.

ACE2 was diluted from stock in 1×PBS to 1 μg/ml and 10 μl aliquots were applied to each working electrode on the PCB and left to incubate for 1 hour at room temperature. Following incubation, the PCBs were rinsed with 1×PBS (10 second water bottle flow per electrode) and dried with compressed air.

Protein target detection. To investigate evidence of specific binding between ligand (ACE2) and protein (HRP conjugated SARS-CoV-2 spike protein) a series of dilutions of the positive control HRP conjugated SARS-CoV-2 spike protein and negative controls of similar sized proteins (HRP conjugated streptavidin and IL-6) were incubated at room temperature for 30 minutes on the PCB sensor arrays with rinsing with 1×PBS (10 seconds water bottle flow per electrode) and EIS measurements between each concentration incubation. HRP conjugated SARS-CoV-2 spike protein and IL-6 concentrations used were 1, 10, 50 and 100 ng/ml (all dilutions in 1×PBS). HRP conjugated streptavidin was obtained as part of an ELISA kit and the concentration was not disclosed. The accompanying instructions recommended a 1:40 dilution for ELISA assays. The series of dilutions used (1:100, 1:75, 1:50, 1:25 and 1:5) were distributed about the 1:40 recommended dilution.

Inactivated virus detection. For detection of inactivated virus a clinical molecular standards kit for SARS-CoV-2 (Qnostics) was purchased. The kit contained positive and negative samples of the virus present in a complex “transport medium” representative of a clinical sample. A series of dilutions of the positive control (inactivated virus+transport medium and human cells) was incubated for 30 mins at room temperature on the PCBs. The concentrations used were 10², 10³, 10⁴, 10⁵ and 10⁶ dC/ml (digital copies per ml). Due to small volume of solutions provided, the negative control (transport medium+human cells) was incubated twice for 30 minutes at room temperature. Room temperature incubations were chosen to replicate the operational environmental conditions likely required for a diagnostic device. The PCBs were rinsed with 1×PBS (10 seconds wash bottle flow per electrode) and EIS measurements preformed between each incubation.

EIS parameters. All EIS measurements used the following parameters. E_{ac}=0.01 V rms, E_{dc}=0 V, frequency range=100 kHz to 1 Hz with 50 frequencies at 9.8/decade and measurements were made versus the open circuit potential (OCP). All measurements were obtained using 5 mM K₃[Fe(CN)₆]/K₄[Fe(CN)₆] in 1×PBS.

Results and Discussion

Fluorocarbon SAM functionalisation. Commonly electrochemical biosensors will have their probe molecule directly attached to the sensor surface (via covalent bonding, physisorption and chemisorption) and surrounded by a hydrocarbon-based SAM. Less commonly the hydrocarbon SAM is immobilized first, and the biomolecule adsorbed into it via hydrophobic physisorption interactions, which can effect better orientation of the probe biomolecule, increasing the likelihood of receptor-target binding. Such an approach does however have the disadvantage in also being a weaker immobilization method than covalent attachment and therefore a higher probability of removal during incubations and wash steps. The investigators sought to consider the use of fluorocarbons as they can offer greatly increased amphiphobicity (hydrophobic and lipophobic character) over hydrocarbons offering stronger physisorption and anti-biofouling properties¹⁷. The ability of fluorocarbons to form a SAM on the PCB electrode surfaces was investigated. An overnight incubation of 1 mM PFDT affected an increase in the measured impedance of the electrodes, which is evident as a larger R d semi-circle (SAM) compared to the clean impedance in the Nyquist plot (FIG. 2A). Quantitatively this can be seen as a mean percentage increase in R d of 928% in (FIG. 2B and in FIG. 2C) with the clean electrodes having a mean R_{ct}=2.5 kΩ and the SAM stage R_{ct}=13 kΩ.

$$Δ \% = \frac{{R}_{c t - A f t e r} - {R}_{c t - B e f o r e}}{{R}_{c t - B e f o r e}} \qquad ( Eq . 1 )$$

Percentage change was calculated using Equation 1, where A % is percentage change, R_{ct-Before} is the R_{ct} of the initial stage, and R_{ct-After} is the R_{ct} of the incubation stage. Significant differences were gauged from the box plots. If the median of one group lies outside the inter quartile range (IQR) of another it is likely there is a significant difference between the groups. T-test analysis was not performed as these experiments were not designed with hypothesis testing in mind and as such may report false results. The box plot (FIG. 2C) showed the clean and SAM stages are likely significantly different as the IQR of both groups do not overlap therefore the PFDT layer formed during SAM formation caused a significant increase in the impedance of the electrodes, providing strong evidence for formation of a layer of immobilised PFDT. This was hypothesized to be due to the well-established process of SAM formation with the SAM molecules attaching to the surface and forming a well-ordered layer on the surface. Such a layer restricts the amount or rate at which the redox active Fe(CN)₆^{3-/4-}ions in the measurement buffer can undergo redox reactions, giving rise to an increase in the impedance measurement. In summary these data showed that a fluorocarbon SAM was successfully formed on the PCB electrodes.

ACE2 hydrophobic immobilisation. A further benefit of the strongly hydrophobic fluorous SAM is that it provides an ideal environment to facilitate hydrophobic physisorption of ACE2 biomolecules. To test this ACE2 protein was incubated in the presences of the electrode SAM. After 1 hour of incubation with 1 μg/ml of ACE2 solution on the SAM functionalized electrodes, a small impedance increase was apparent (FIG. 2A). In absolute terms this was a further 2 kΩ Rct increase over the SAM alone (FIG. 2C). This was indicative of hydrophobic physisorption of the enzyme into the supporting SAM layer. The ACE2 electrodes were significantly different from the clean group but not from the SAM group. This finding was not entirely unexpected as the fluorous SAM had covered a previously clean surface with a densely packed layer resulting in a large impedance change. ACE2 has added to this layer by adsorbing within the fluorous SAM, further blocking the electrode surface; however, the relatively low number of ACE2 molecules in comparison to the fluorous molecules present on the surface accounts for the small relative change in the impedance. Having demonstrated successful assembly of the PFDT SAM and having seen evidence of ACE2 incorporation into the SAM structure a series of ligand binding experiments were undertaken next.

HRP conjugated SARS-CoV-2 spike protein (positive) and HRP conjugated streptavidin protein (negative). Having confirmed successful immobilisation of ACE2, HRP conjugated SARS-CoV-2 spike protein (HRP conjugated version was used to enable visual determination of binding) was incubated with the functionalised sensor surface for 30 minutes. The measured impedance for 1, 10, 50 and 100 ng/ml consistently increased compared to the preceding concentration demonstrating dose dependant behaviour (FIG. 3A). The mean percentage change of R_{ct} (n=4) ranged from 96% at the lowest concentration to 156% at the highest concentration (FIG. 3B, red). This showed the HRP conjugated SARS-CoV-2 spike protein had bound to the PFDT-ACE2 modified sensor. The addition of a diluted series of HRP conjugated streptavidin (negative control 1:100, 1:75, 1:50, 1:25 and 1:5) allowed for the confirmation of specific binding of HRP conjugated SARS-CoV-2 spike protein. The mean percentage change of Rd (n=4) for the negative control ranged from 6.2% at the lowest concentration to 52.8% for the highest (FIG. 3B, blue). The negative response appeared to plateau with two consecutive percentage change measurements for 1:25 (53.1%) and 1:5 (52.8%) and two similar data spreads and values (FIG. 2C, blue). All normalised data in these experiments used the ACE2 signal as the normalising factor. There were likely significant differences between the ACE2 and all the positive control concentrations further evidencing strong HRP conjugated SARS-CoV-2 spike protein binding (FIG. 2C, red). The negative control experiments were not significantly different along the dilution series indicating weak binding to the PFDT-ACE2 modified sensor. All positive groups are likely significantly different from all negative groups indicated by the median of the negative data lying outside the IQR of the positive groups. Considering this evidence, it was concluded that the positive HRP conjugated SARS-CoV-2 spike protein successfully and specifically bound to the ACE2 receptor whilst the HRP conjugated streptavidin did not specifically bind. The signal generated by the negative control was most likely due to a small amount of absorption into the fluorous SAM. The signals are low in comparison to the positive control and appear to saturate at a low level, suggesting that the fluorous SAM layer provided anti-biofouling properties allowing for the positive signal to dominate. It should also be pointed out that the starting concentration of the HRP labelled streptavidin solution was in the region of 1 mg/mL meaning the dilutions series of negative control protein solutions was significantly more concentrated (at least one order of magnitude) than the HRP conjugated spike protein solutions. The fact that there is strong evidence of specific binding of the positive and comparatively weaker binding of the negative also confirms that ACE2 is physisorbed into the fluorous SAM in significant enough quantity and orientation to bind the positive ligand. If ACE2 was bound in an unfavourable orientation, ligand access to receptor binding sites would have been hindered and greatly reduced the positive signal.

It was also observed (FIG. 6) that binding efficiency was significantly reduced when using the shorter eight carbon octane-thiol and longer eleven chain undecanethiol. This further indicates that the strong hydrophobic character of the PFDT layer produces an adsorption mechanism responsible for the sensor behaviour. Since an HRP label was employed for the positive protein sample, it was prudent to also use an HRP labelled negative control to account for the potential of HRP to contribute to the binding signal. HRP conjugated SARS-CoV-2 spike protein is approximately 154 kDa and HRP conjugated streptavidin is approximately 104 kDa. The two proteins were thus of relative similar size and both containing the HRP label allowed for good comparison between the two. An indication of the Y-axis limit of detection (LOD) was calculated using Equation 2;

P_{LOD}=Yᵢ+3SDᵢ (Eq. 2)

where Y_{LOD} was the limit of detection of the Y-axis parameter (normalized R_{ct}), Yᵢ was the y-intercept value obtained from linear regression of the data and SDᵢ was the accompanying standard deviation of the y-intercept. The value obtained obtained for the normalised R_{ct} Y_{LOD} for SARS-CoV-2 HRP conjugated spike protein was 2.1 (FIG. 3D). The lowest concentration signal tested was at the threshold of this limit. All other concentrations were above the limit. A limit of detection for the X-axis was also calculated from the linear regressed data (R²=0.99342). Using the Y LOD and the equation of the fitted line gave an X_{LOD} of 1.06 ng/ml for SAR-CoV-2 HRP conjugated spike protein.

HRP conjugated SARS-CoV-2 spike protein (positive) versus IL-6 (negative). A second negative control was investigated using the protein IL-6 (26 kDa) with equal concentrations as used for the positive control (1, 5, 10, 50, 100 ng/ml). IL-6 is a myokine and cytokine common in the human body under normal circumstances, especially after exercise. It has inflammatory and immune effects in a multitude of diseases including bacterial and viral infection. IL-6 has been shown to be present at elevated levels in the ‘cytokine storm’ which is observed in many advanced cases of COVID-19. This would therefore represent a potential source of artefact that could affect specific virus detection and was thus chosen as a negative control. This time, each control group used a single PCB array instead of portioning a single board into positive and negative sections. This increased the amount of collected data for both groups from n=4 to n=8. The HRP conjugated SARS-CoV-2 spike protein response for increasing concentration was once again seen to sequentially increase (FIG. 4A). This was also evident from the R_{ct} percentage change (FIG. 4B, red) ranging from 24.4% at the lowest concentration to 300% at the highest concentration. This again showed HRP conjugated SARS-CoV-2 spike protein had bound to the PFDT-ACE2 complex. The negative control IL-6 showed smaller mean percentage increases with 10 and 50 ng/ml being similar 57% and 59% (FIG. 4B, blue). The means ranged from 14% at the lowest concentration to 77% at the highest. There were likely differences between the positive and negative controls of the 1, 50 and 100 ng/ml concentrations (FIG. 4C). The positive data (FIGS. 4B and 4C), showed the previously seen increasing dose dependant behaviour. The negative data increased slowly in agreement with the small mean percentage changes (FIG. 4B). These results confirmed that the HRP conjugated SARS-CoV-2 spike protein was successfully and specifically detected and that the negative IL-6 signal was suppressed alluding again to anti-biofouling properties arising from the fluorous SAM. It is important to note that the IL-6 concentrations used were 10³ to 10⁵ times higher than the IL-6 levels detected in COVID-19 patients. Patients that progressed to acute respiratory distress syndrome (ARDS) had a median of 7.39 pg/mL²² and patients that died had a median of 11.4 pg/mL²³. This experiment was able to show discrimination with a contamination level far in excess of that seen in clinical COVID-19 samples. The normalized R_{ct} Y_{LOD} was found to be 1.21 (FIG. 4D), which was a slight improvement on that reported in the previous section (Y_{LOD}=2.1). Only the 1 ng/ml concentration data point intersected this limit suggesting that the 1 ng/ml may not be a reliable value for clear detection. The concentration X_{LOD} however was found to be 1.68 ng/ml (R²=0.99).

Inactivated SARS-CoV-2 detection. Having shown that the spike protein ligand was able to specifically bind to the ACE2 receptor in the presence of negative control proteins, the focus changed to virus detection. A dilution of series of inactivated whole virus (10², 10², 10⁴, 10⁵ and 10⁶ dC/ml) was tested against undiluted negative control samples from the same molecular standards kit and containing lysed cells and proteins in the “transport medium” in order to mimic a complex clinical sample. Incubations resulted in a consistently increasing R_{ct} (FIG. 5A). The mean percentage change for the positive control (n=7) ranged from 106% at the lowest to 211% at the second highest concentration (FIG. 5A, red). The highest concentration saw a decrease from 211% to 168%. This is possibly due to removal of the virus or virus+ACE2 complex or a subsequent reordering or desorption of the SAM resulting from the presence of large virus quantities through successive experiments. Similar effects have been seen in other work within our group (unpublished data). The negative control contained the same background transport medium plus human cells as the positive control but lacked the whole virus. Actual concentrations and composition were not provided by the vendor however it was stated that samples were representative of clinical human specimens and quantification data was supplied in the form of digital copies per mL (dC/mL). The first negative sample was applied at the same time as the first positive control and underwent the same treatments. A second negative application was performed at the same time as the second positive. Only two negative treatments were possible due to the sample volume required versus the low volume supplied. Both negative responses showed almost identical mean percentage changes 114.4% and 113.9% (FIG. 5B, blue). This showed that the background solution produces a high signal but one which saturated immediately. In contrast, the signal from samples containing SARS-CoV-2 continued to grow with increasing virus concentration. The normalized data showed that 10² and 10³ dC/ml concentrations were not significantly different from the negative however 10⁴, 10⁵ and 10⁶ dC/ml were likely significantly different (FIG. 5C). This data showed that the virus was specifically bound to the ACE2 receptor and could be distinguished from the negative at 10⁴ dC/ml and above. Clinical levels range from 10⁴ to 10¹¹ RNA copies/ml^{25,26}. This is within the distinguishable region presented. The performance of the sensor itself was indicated by a normalised R_{ct} Y_{LOD} of 1.83 (FIG. 5D). No data points intersected this limit indicating the lowest 10² concentration was a successful detection. The X_{LOD} was 37.8 dC/ml (R²=0.96064). The sensor therefore had the performance to detect over the entire range tested and with the potential to discriminate lower concentrations if the positive to negative signal ratio is improved upon. The results of testing with inactivated virus were highly compelling; first the virus had been heated at 65° C. for 30 mins and gamma irradiated so it's three dimensional structure would have been significantly disrupted, and the positive and negative virus samples were present in a complex medium used to culture the cells which produced the virus and therefore bore a similar resemblance to other biological media such as saliva and serum. The 30 min incubation times provided compelling signal increases meaning the measurement was relatively fast, especially contrasted to the gold standard nucleic acid amplification detection. Finally, there is considerable room for optimisation of the assay protocol, for example, shortening of the viral incubation step and optimisation of washing procedures to maximise discriminatory power.

### CONCLUSIONS

The preparation and testing of a simple and easily produced electrochemical biosensor for SARS-CoV-2 has been demonstrated. The sensor consists of a base SAM composed entirely of PFDT with ACE2 hydrophobically absorbed into the layer. It was possible, using solutions of HRP-conjugated spike protein (positive) and HRP conjugated streptavidin and IL-6 (negatives) to detect the viral spike protein in a sensitive, specific and dose dependant manner. Detection and discrimination of inactivated SARS-CoV-2 virus present in a complex medium (cell culture lysate) was demonstrated to confirm the sensitivity, specificity and resistance to biological fouling necessary for a useful biosensor for SARS-CoV-2. The ease with which the sensor can be prepared and the compatibility of the preparation steps with mass manufacturing techniques mean the assay is potentially adoptable on existing commercial biosensor formats. This would allow for wide distribution of point of care assays for rapid testing of the population with diagnostics being at the centre of test, track and tracing of contacts, central to efforts to control the COVID-19 pandemic.

The presented sensor uses EIS to detect binding from solutions of recombinant SARS-CoV-2 spike protein and positive and negative samples of inactivated SARS-CoV-2 from a fully validated molecular standards kit. Advantages of the sensor design are that the result can be produced in a label free manner (i.e. there is no need to add a fluorescent or electrochemical label during the assay steps), the test is designed to measure viral particles in saliva so there is no chance of detecting residual viral RNA post infection and crucially the sensor has been designed for ease of upscaling and manufacture with two simple production steps: (1) facile SAM formation and (2) ACE2 functionalisation. In the work, the assay is demonstrated on a low cost eight working electrode PCB sensor system, however, the assay can be transferred onto even more mass manufacturable platforms such as screen-printed devices or glucose format test strips. Importantly, this would unlock integration with a well-established high volume production environment and lead to a diagnostic with the potential for widespread, rapid, point of need use.

### REFERENCES

(1) Cesewski, E.; Johnson, B. N. Electrochemical Biosensors for Pathogen Detection. Biosens. Bioelectron. 2020, 159, 112214. https://doi.org/10.1016/j.bios.2020.112214.
(2) Newman, J. D.; Setford, S. J. Enzymatic Biosensors. Mol. Biotechnol. 2006, 32 (3),249-268. https://doi.org/10.1385/MB:32:3:249.
(3) Turner, A. P. F. Biosensors: Sense and Sensibility. Chem. Soc. Rev.2013, 42 (8), 3184-3196. https://doi.org/10.1039/c3cs35528d.
(4) Manzano, M.; Viezzi, S.; Mazerat, S.; Marks, R. S.; Vidic, J. Rapid and Label-Free Electrochemical DNA Biosensor for Detecting Hepatitis A Virus. Biosens. Bioelectron. 2018, 100 (Supplement C),89-95. https://doi.org/10.1016/j.bios.2017.08.043.
(5) Tong, Y.; Li, H.; Guan, H.; Zhao, J.; Majeed, S.; Anjum, S.; Liang, F.; Xu, G. Electrochemical Cholesterol Sensor Based on Carbon Nanotube@molecularly Imprinted Polymer Modified Ceramic Carbon Electrode. Biosens. Bioelectron. 2013,47,553-558. https://doi.org/10.1016/j.bios.2013.03.072.
(6) Jiang, H.; Sun, A.; Venkatesh, A. G.; Hall, D. A. An Audio Jack-Based Electrochemical Impedance Spectroscopy Sensor for Point-of-Care Diagnostics. IEEE Sens. J. 2017, 17 (3), 589-597. https://doi.org/10.1109/JSEN.2016.2634530.
(7) Kim, Y.-R.; Bong, S.; Kang, Y.-J.; Yang, Y.; Mahajan, R. K.; Kim, J. S.; Kim, H. Electrochemical Detection of Dopamine in the Presence of Ascorbic Acid Using Graphene Modified Electrodes. Biosens. Bioelectron. 2010, 25 (10), 2366-2369. https://doi.org/10.1016/j.bios.2010.02.031.
(8) Bard, A.; Faulkner, L. Electrochemical Methods: Fundamentals and Applications, 2nd ed.; Wiley, 2001.
(9) Bahadir, E. B.; SezgintOrk, M. K. A Review on Impedimetric Biosensors. Artif. Cells Nanomedicine Biotechnol. 2016, 44 (1),248-262. https://doi.org/10.3109/21691401.2014.942456.
(10) Yang, Z.; Gonzalez-Cortes, A.; Jourquin, G.; Viré, J.-C.; Kauffmann, J.-M.; Delplancke, J.-L. Analytical Application of Self Assembled Monolayers on Gold Electrodes: Critical Importance of Surface Pretreatment. Biosens. Bioelectron. 1995, 10 (9), 789-795. https://doi.org/10.1016/0956-5663(95)99217-9.
(11) Guo, L.-H.; Facci, J. S.; McLendon, G.; Mosher, R. Effect of Gold Topography and Surface Pretreatment on the Self-Assembly of Alkanethiol Monolayers. Langmuir 1994, 10 (12), 4588-4593. https://doi.org/10.1021/Ia00024a033.
(12) Bain, C. D.; Troughton, E. B.; Tao, Y. T.; Evall, J.; Whitesides, G. M.; Nuzzo, R. G. Formation of Monolayer Films by the Spontaneous Assembly of Organic Thiols from Solution onto Gold. J. Am. Chem. Soc. 1989, 111 (1), 321-335. https://doi.org/10.1021/ja00183a049.
(13) Chapman, R. G.; Ostuni, E.; Takayama, S.; Holmlin, R. E.; Yan, L.; Whitesides, G. M. Surveying for Surfaces That Resist the Adsorption of Proteins. J. Am. Chem. Soc. 2000, 122 (34), 8303-8304. https://doi.org/10.1021/ja000774f.
(14) Xu, D.; Su, Y.; Zhao, L.; Meng, F.; Liu, C.; Guan, Y.; Zhang, J.; Luo, J. Antibacterial and Antifouling Properties of a Polyurethane Surface Modified with Perfluoroalkyl and Silver Nanoparticles. J. Biomed. Mater. Res. A 2017, 105 (2), 531-538. https://doi.org/10.1002/jbm.a.35929.
(15) Cai, Q. J.; Chan-Park, M. B.; Zhou, Q.; Lu, Z. S.; Li, C. M.; Ong, B. S. Self-Assembled Monolayers Mediated Charge Injection for High Performance Bottom-Contact Poly(3,3″-Didodecylquaterthiophene) Thin-Film Transistors. Org. Electron.2008, 9(6),936-943. https://doi.org/10.1016/j.orgel.2008.06.014.
(16) Flynn, G. E.; Withers, J. M.; Macias, G.; Sperling, J. R.; Henry, S. L.; Cooper, J. M.; Burley, G. A.; Clark, A. W. Reversible DNA Micro-Patterning Using the Fluorous Effect. Chem. Commun. 2017, 53 (21),3094-3097. https://doi.org/10.1039/C7CC00288B.
(17) Cametti, M.; Crousse, B.; Metrangolo, P.; Milani, R.; Resnati, G. The Fluorous Effect in Biomolecular Applications. Chem.Soc. Rev. 2012, 41 (1), 31-42. https://doi.org/10.1039/c1cs15084g.
(18) Antibody Adsorption and Orientation on Hydrophobic Surfaces. Wiseman & Frank https://pubs.acs.org/doi/10.1021/Ia203095p
(19) Towler, P.; Staker, B.; Prasad, S. G.; Menon, S.; Tang, J.; Parsons, T.; Ryan, D.; Fisher, M.; Williams, D.; Dales, N. A.; Patane, M. A.; Pantoliano, M. W. ACE2 X-Ray Structures Reveal a Large Hinge-Bending Motion Important for Inhibitor Binding and Catalysis. J. Biol. Chem. 2004, 279 (17), 17996-18007.https://doi.org/10.1074/jbc.M311191200.
(20) Zhou, T.; Tsybovsky, Y.; Olia, A. S.; Gorman, J.; Rapp, M.; Cerutti, G.; Chuang, G.-Y.; Katsamba, P. S.; Nazzari, A.; Sampson, J. M.; Schein, A.; Wang, P.; Bimela, J.; Shi, W.; Teng, 1.-T.; Zhang, B.; Boyington, J. C.; Sastry, M.; Stephens, T.; Stuckey, J.; Wang, S.; Friesner, R. A.; Ho, D. D.; Mascola, J. R.; Shapiro, L.; Kwong, P. D. Cryo-EM Structures Delineate a PH-Dependent Switch That Mediates Endosomal Positioning of SARS-CoV-2 Spike Receptor-Binding Domains. bioRxiv 2020, 2020.07.04.187989. https://doi.org/10.1101/2020.07.04.187989.
(21) Fairhead, M.; Krndija, D.; Lowe, E. D.; Howarth, M. Plug-and-Play Pairing via Defined Divalent Streptavidins. J. Mol. Biol. 2014, 426 (1),199-214. https://doi.org/10.1016/j.jmb.2013.09.016.
(22) Wu, C.; Chen, X.; Cai, Y.; Xia, J.; Zhou, X.; Xu, S.; Huang, H.; Zhang, L.; Zhou, X.; Du, C.; Zhang, Y.; Song, J.; Wang, S.; Chao, Y.; Yang, Z.; Xu, J.; Zhou, X.; Chen, D.; Xiong, W.; Xu, L.; Zhou, F.; Jiang, J.; Bai, C.; Zheng, J.; Song, Y. Risk Factors Associated With Acute Respiratory Distress Syndrome and Death in Patients With Coronavirus Disease 2019 Pneumonia in Wuhan, China. JAMA Intern. Med. 2020, 180 (7), 934-943. https://doi.org/10.1001/jamainternmed.2020.0994.
(23) Coomes, E. A.; Haghbayan, H. Interleukin-6 in COVID-19: A Systematic Review and Meta-Analysis. medRxiv 2020, 2020.03.30.20048058. https://doi.org/10.1101/2020.03.30.20048058.
(24) Ruan, Q.; Yang, K.; Wang, W.; Jiang, L.; Song, J. Clinical Predictors of Mortality Due to COVID-19 Based on an Analysis of Data of 150 Patients from Wuhan, China. Intensive Care Med. 2020, 1-3. https://doi.org/10.1007/s00134-020-05991-x.
(25) Xu, G.-Y.; Yu, H.-A.; Hong, J.; Stahl, M.; McDonagh, T.; Kay, L. E.; Cumming, D. A. Solution Structure of Recombinant Human Interleukin-611Edited by P. E. Wright. J. Mol. Biol. 1997,268 (2),468-481. https://doi.org/10.1006/jmbi.1997.0933.
(26) Bar-On, Y. M.; Flamholz, A.; Phillips, R.; Milo, R. SARS-CoV-2 (COVID-19) by the Numbers. eLife 2020, 9, e57309. https://doi.org/10.7554/eLife.57309.
(27) Wölfel, R.; Corman, V. M.; Guggemos, W.; Seilmaier, M.; Zange, S.; Müller, M. A.; Niemeyer, D.; Jones, T. C.; Vollmer, P.; Rothe, C.; Hoelscher, M.; Bleicker, T.; Brünink, S.; Schneider, J.; Ehmann, R.; Zwirglmaier, K.; Drosten, C.; Wendtner, C. Virological Assessment of Hospitalized Patients with COVID-2019. Nature 2020, 581 (7809), 465-469. https://doi.org/10.1038/s41586-020-2196-x.

1. An electrochemical biosensor for use in detecting a target analyte, the sensor comprising:
at least one detection electrode comprising a surface coated with a self-assembled monolayer (SAM), wherein the SAM comprises, consists essentially of, or consists of a hydrofluorocarbon or fluorocarbon molecule bound to the surface of the electrode through a reactive sulphur or silicon group present on the hydrofluorocarbon or fluorocarbon.

2. The electrochemical biosensor according to claim 1, wherein the hydrofluorocarbon or fluorocarbon molecule is a linear, branched or cyclic alkane, alkene or alkyne molecule having a single or multiple reactive sulphur or silicon groups.

3. The electrochemical biosensor according to claim 1 or 2, wherein the reactive sulphur group(s) is/are a thiol or silane groups(s).

4. The electrochemical biosensor according to any preceding claim, wherein the fluorocarbon molecule is a linear fluoro alkanethiol, or fluoro alkanesilane.

5. The electrochemical biosensor according to claim 4, wherein the linear fluoro alkanethiol, or fluoro alkanesilane is selected from the group consisting of:
1H,1H,2H,2H-Perfluorodecanethiol
3,3,4,4,5,5,6,6,7,7,8,8,8-Tridecafluoro-1-octanethiol
3,3,4,4,5,5,6,6,7,7,8,8,9,9,10,10,10-Heptadecafluoro-1-decanethiol
3,3,4,4,5,5,6,6,6-Nonafluoro-1-hexanethiol
2,2,2-Trifluoroethanethiol
1H,1H,2H,2H-Perfluorooctyltriethoxysilane
1H,1H,2H,2H-Perfluorodecyltriethoxysilane; and
1H,1H,2H,2H-Perfluorododecyltrichlorosilane.

6. The electrochemical biosensor according to claim 4 wherein the linear fluoro alkanethiol is 1H,1H,2H,2H-Perfluorodecanethiol.

7. The electrochemical biosensor according to any preceding claim, wherein the electrode surface is formed from a glassy carbon; metal oxide; conducting polymer; or noble metal including gold, ruthenium, rhodium, palladium, platinum and silver.

8. The electrochemical biosensor according to claim 7 wherein the electrode surface is gold.

9. An electrochemical biosensor according to any preceding claim, further comprising a biological agent captured by the SAM layer coated on the surface of the electrode.

10. The electrochemical biosensor according to claim 9 wherein the biological agent is captured by physisorption to the SAM layer.

11. A method of making an electrochemical biosensor according to any of claims 1-8, the method comprising:
forming a SAM on a surface of at least one detection electrode, by contacting the surface of the at least one detection electrode with a solution comprising an organic solvent and a hydrofluorocarbon or fluorocarbon molecule as described hereinabove, and
allowing the solvent to evaporate and the SAM to form on the surface of the at least one detection electrode.

12. The method according to claim 11, further comprising:
contacting the SAM coated electrode with a solution comprising the biological agent and allowing the biological agent to be captured by the SAM layer coated on the electrode.

13. The electrochemical biosensor according to any of claims 1-10 further comprising at least one reference and/or counter electrode electrically coupled to said at least one detection electrode.

14. The electrochemical biosensor according to any of claims 1-10, wherein the electrodes are provided on a substrate.

15. The electrochemical biosensor according to claim 14 provided in the form of screen printed electrodes; microelectrodes; on a printed circuit board; or on FETS/OFETS.

16. Use of an electrochemical biosensor according to any of claims 1-10 and 14-15 in the detection of a target analyte, which is capable of binding, typically specifically binding, to the biological agent captured by the sensor.

17. Use according to claim 16, wherein the target analyte is a chemical (such as a hormone, narcotic or pollutant) or biological molecule (such as, a peptide, protein, glycoprotein, enzyme, glycolipid, cell surface receptor, cytokine, antibody, or nucleic acid).

18. Use according to claim 16 or 17 wherein the target analyte is free within the sample being analyzed, or is still be part of a cell, cell membrane, virus coat, in which the target analyte, is normally found in situ.

19. Use according to claim 18 wherein the target analyte is a virus coat protein.

20. Use according to claim 16, wherein the biological macromolecule, which is captured by the electrochemical biosesnor is ACE-2 and the target analyte is SARS-CoV-2 or a coat protein thereof.

21. Use according to claim 20, wherein the SARS-CoV-2 is COVID-19.
